(19) United States
(12) Patent Application Publication
(10) Pub. No.: US 2011/0106069 A1
(43) Pub. Date: May 5, 2011

(54) LASER FOCUSING TIP PROTECTION

(76) Inventor: Robert J. Brooks, Clinton, WA (US)

(21) Appl. No.: 12/915,932

(22) Filed: Oct. 29, 2010

Related U.S. Application Data
(60) Provisional application No. 61/255,969, filed on Oct. 29, 2009.

(51) Int. Cl. A61B 18/20

(52) U.S. Cl. 606/14

(57) ABSTRACT

A laser tip has at least a portion of its surface as a more easily cleaned surface. In some embodiments, the more easily cleaned surface may be formed integrally with the laser tip. In some embodiments, the more easily cleaned surface may be a coating or sheath that covers the exposed exterior surface and can be removed after surgery for cleaning or disposal. The more easily cleaned surface may include a lubricious coating which may prevent or minimize the adherence of charred matter on the tip.

### CROSS-REFERENCE TO RELATED APPLICATIONS

This application claims the benefit of priority of U.S. Provisional patent application No. 61/255,969, filed Oct. 29, 2009, which is herein incorporated by reference.

### BACKGROUND OF THE INVENTION

The present invention relates to laser tips and, more particularly, to a laser focusing/delivery tip having means for minimizing adherence of material, such as charred tissue, therefrom.

Carbon dioxide lasers, and other wavelength lasers, are used to incise, excise and ablate soft tissue in medical, dental and veterinary procedures. A common type of these lasers uses a flexible waveguide to deliver the energy to the treatment area. The surgeon selects a tip that gets inserted into a handpiece at the end of the flexible waveguide. Tip selection includes consideration for length, shape and effective spot size of the delivered laser energy. For economic reasons, many surgeons chose to clean, re-sterilize and reuse tips for multiple procedures.

The tip is subjected to contamination generated during the reaction of the treatment tissue and the laser energy, with the exposed exterior of the tip receiving much of the spattered material.

Adherence of charred spatter from laser beam interaction with tissue being treated often causes build-up on the exit port of a laser. This build-up may affect the laser beam quality. The exterior surface of the laser focusing tip retains the spattered material and must be removed during post op cleaning for re-sterilization.

As can be seen, there is a need for an improved tip design that minimizes adherence of the spattered particles and facilitates removal of the spattered materials from the exterior of the tip.

### SUMMARY OF THE INVENTION

In one aspect of the present invention, a laser tip comprises an insertion end and a delivery tip; and a lubricous surface formed on at least a portion of the delivery tip, wherein the insertion end is adapted to fit into a handpiece of a laser.

These and other features, aspects and advantages of the present invention will become better understood with reference to the following drawings, description and claims.

### BRIEF DESCRIPTION OF THE DRAWINGS

FIG. 1 is a side view of a laser tip according to an exemplary embodiment of the present invention;

FIG. 2 is a side view of a different sized laser tip according to an exemplary embodiment of the present invention;

FIG. 3A is a side view of the laser tip of FIG. 2 fully inserted into a handpiece;

FIG. 3B is a side view of the laser tip of FIG. 2 partially inserted into the handpiece;

FIG. 4A is a side view of a laser tip according to an alternate embodiment of the present invention fully inserted into a handpiece; and

FIG. 4B is a side view of the laser tip of FIG. 4A partially inserted into the handpiece.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, an embodiment of the present invention provides a laser tip having at least a portion of its surface as a more easily cleaned surface. In some embodiments, the more easily cleaned surface may be formed integrally with the laser tip. In some embodiments, the more easily cleaned surface may be a coating or sheath that covers the exposed exterior surface and can be removed after surgery for cleaning or disposal. The more easily cleaned surface may include a lubricious coating which may prevent or minimize the adherence of charred matter on the tip.

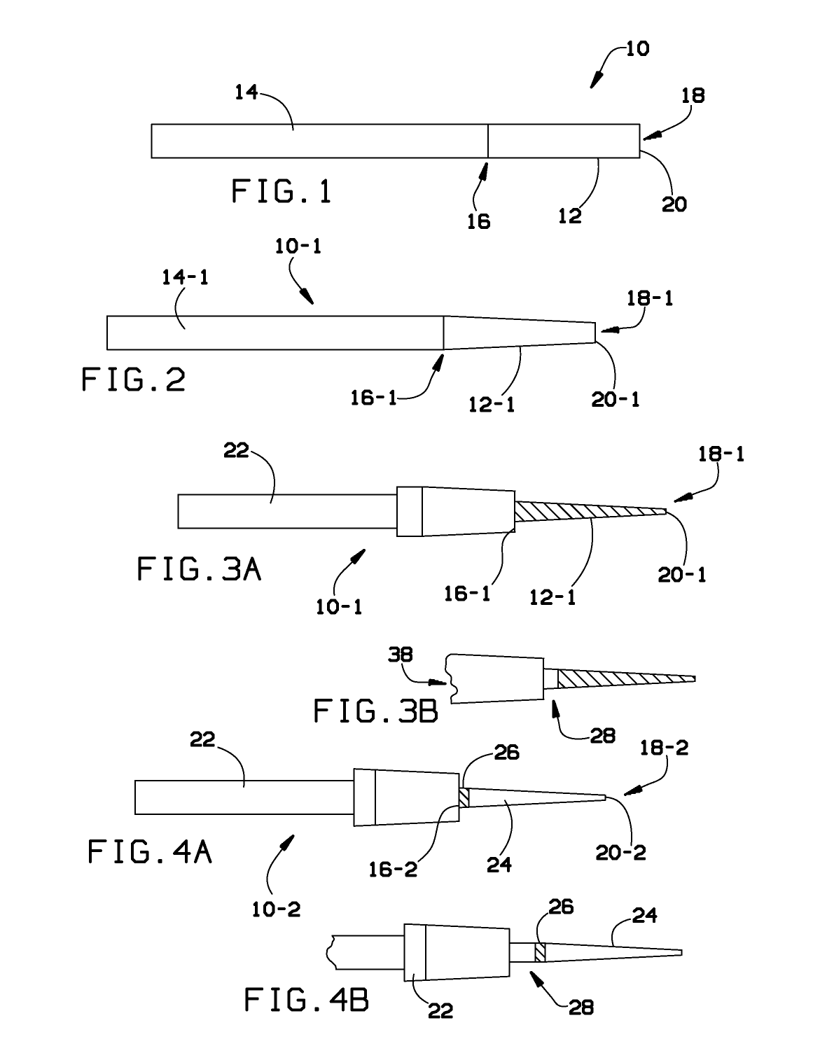

Referring to FIG. 1, there is shown a laser tip 10 having a delivery tip 12, which may be a coated tip 12, and an insertion end 14. The insertion end 14 is adapted for insertion into an end of a handpiece 22 of a flexible waveguide (see FIG. 3). The coated tip 12 may include a lubricious coating or surface applied to or part of the laser tip 10. The lubricious coating or surface may extend up to an insertion point 16. The insertion point 16 provides an indication of how far the insertion end 14 should be inserted into the handpiece 22. The insertion point 16 may allow users a way to accurately and consistently insert the laser tip 10 for use. The laser tip 10 may have a laser delivery end 20 including an opening 18 for directed laser energy. The laser tip 10 may be produced using a variety of materials, including metal, ceramic, plastic and the like.

The lubricious coating can be applied to the exterior surfaces of the tip 12 or the tip 12 may include a lubricious material as part of its structure. The lubricious coating may be color-coded to provide a method of identifying the size of the opening 18. This size dictates the effective ‘spot size’ of laser energy when applied to the surgical site. For example, a 1.0 mm opening may be blue, a 0.5 mm opening may be green, and a 0.25 mm opening may be purple. The lubricious surface may be applied to the tip 12 via conventional methods, including, for example, as a spray coating, as an additive to the material used to form the tip, or as a jacket applied to the exterior of the delivery tip. In some embodiments, for example, with spray coating, the lubricious surface may cover not only the exterior surface of the tip 12, but also the tip end 20 of the tip 12. In some embodiments, a small amount of lubricious surface may cover a portion of the inside surface of the tip 12. For example, the lubricious surface may extend, from the opening 18, onto the interior surface of the tip 12, a distance related to the size of the opening 18. In some embodiments, the lubricious surface may extend a distance inside the tip 12 from about 0.1 to about 1.0 times the diameter of the opening 18. For example, for a 0.5 mm opening, the lubricious surface may extend from about 0.05 mm to about 0.50 mm inside the tip 12.

Referring to FIGS. 2 and 3A, a laser tip 10-1 is shown with smaller diameter opening 18-1 (as compared to opening 18 of FIG. 1). Similar to the embodiment of FIG. 1, the laser tip 10-1 includes an insertion end 14-1, a coated tip 12-1, an insertion indicator 16-1, and a tip end 20-1. The side view of FIG. 3A shows the laser tip 10-1 inserted into the handpiece 22. The side view of FIG. 3B shows the laser tip 10-1 not fully inserted into the handpiece 22.

Referring now to FIG. 4A, in an alternate embodiment of the present invention, the laser tip 10-2 may include a tip 24. The tip 24 may or may not contain a lubricious coating. A colored stripe 26 may be formed at the insertion point 16-2 of the laser tip 10-2. Such a laser tip 10-2 may provide a user with an accurate and consistent indication of full and proper insertion use of the laser tip 10-2. FIG. 4B shows the laser tip 10-2 not fully inserted into the handpiece 22. In some embodiments, the tip 24 may be coated as described above. Similar to the above described embodiments, laser tip 10-2 may include an opening 18-2 at a tip end 20-2 thereof.

During surgery, the adhering spattered material may be wiped from the exterior surfaces of the delivery tip. After surgery, the surgical assistant may disinfect and clean the tip of any foreign matter prior to re-sterilization.

The lubricious surface may be made from, for example, a polytetrafluoroethylene material, such as Teflon®. Other coatings, surfaces, additives, and the like, may be used, provided they provide a delivery tip that may be wiped clean, for example, during surgery.

It should be understood, of course, that the foregoing relates to exemplary embodiments of the invention and that modifications may be made without departing from the spirit and scope of the invention as set forth in the following claims.

What is claimed is:

1. A laser tip comprising:
an insertion end and a delivery tip; and
a lubricous surface on at least a portion of the delivery tip, wherein
the insertion end is adapted to fit into a handpiece of a laser.

2. The laser tip of claim 1, wherein the lubricious surface is a coating formed on the delivery tip.

3. The laser tip of claim 2, wherein the coating is color-coded to indicate a diameter of an opening in an end of the delivery tip.

4. The laser tip of claim 3, wherein the coating is a polytetrafluoroethylene material.

5. The laser tip of claim 1, wherein the lubricious surface is applied on the delivery top and is not applied to an insertion end, thereby providing an insertion indicator on the laser tip.

6. The laser tip of claim 1, further comprising a colored ring formed around the circumference of the laser tip, the colored ring defining an insertion depth for the insertion end.

7. The laser tip of claim 2, wherein the coating is a sheath that covers the exposed exterior surface of the delivery tip.

8. The laser tip of claim 1, wherein the lubricious surface covers a tip end of the laser tip.

9. The laser tip of claim 1, wherein the lubricious surface covers a portion of an inside surface of the delivery tip.

10. The laser tip of claim 1, wherein the delivery tip includes a lubricious material as part of its structure, the lubricious material providing an exterior surface of the delivery tip that may be wiped clean.
